(19) United States
(12) Patent Application Publication
(10) Pub. No.: US 2016/0198705 A1
(43) Pub. Date: Jul. 14, 2016

(54) APPLICATION OF BISPHENOL SALT IN PREPARING DISINFECTANT USED FOR STERILIZATION OR FORMALDEHYDE REMOVAL

(71) Applicant: NINGBO YUCHEN ENVIROCLEAN TECH CO., LTD., NINGBO, ZHEJIANG PROVINCE (CN)

(72) Inventors: ZUOLIN ZHU, NINGBO, ZHEJIANG PROVINCE (CN); MENG SUN, NINGBO, ZHEJIANG PROVINCE (CN); ZHENJI ZHU, NINGBO, ZHEJIANG PROVINCE (CN); KANGFU GU, NINGBO, ZHEJIANG PROVINCE (CN); SHANQING SUN, NINGBO, ZHEJIANG PROVINCE (CN); TONGXING XIE, NINGBO, ZHEJIANG PROVINCE (CN); JIABAO ZHAO, NINGBO, ZHEJIANG PROVINCE (CN); WENHAI WANG, NINGBO, ZHEJIANG PROVINCE (CN)

(21) Appl. No.: 14/914,923

(22) PCT Filed: May 22, 2014
(86) PCT No.: PCT/CN2014/078084

§ 371 Date: Feb. 26, 2016

(30) Foreign Application Priority Data
Sep. 7, 2013 (CN) 201310405329.1

(51) Int. Cl. A01N 31/08; A61L 9/14; A01N 33/24; A01N 37/40; A01N 41/04

(52) U.S. Cl. CPC A01N 31/08; A01N 37/40; A01N 41/04; A01N 33/24; A61L 9/145

(57) ABSTRACT

The present invention relates to an application of bisphenol salts in preparing a disinfectant used for sterilization or formaldehyde removal. The present invention provides a sterilization and disinfection system that uses oxygen in the air as oxidizer, kills microorganisms via catalyzed oxidation, and is particularly suitable in an air purification system. The disinfectant of the present invention can be used for an extended period of time insofar that air is continuously introduced, obviates the need for an anti-viral agent, also obviates the need for a peroxide that is potentially hazardous to safety, uses no halogen-containing irritating substance, and use no alcohol having risk of explosion. This disinfectant system that uses no precious metal is easy to prepare, low in use cost, and free of secondary pollution, and provides a good application prospect.

### FIELD OF THE INVENTION

The present invention relates to an application of bisphenol salt in preparing disinfectant used for sterilization or formaldehyde removal. The present invention provides a sterilization and disinfection system that use oxygen in the air as an oxidizer, killing microorganisms via catalyzed oxidation, and is particularly suitable for an air purification system.

### BACKGROUND OF THE INVENTION

Improving air quality, particularly removing the pathogenic microorganisms in the air, via air purification measure is one of the methods that are worth vigorously promoting in the case of current continuous increase of population density, substantial promotion of industrialization, gradual worsening of environmental pollution, particularly air pollution, severely influencing human health.

A brand new concept firstly proposed by the inventors in the world wherein oxygen in the air is activated by chemical catalysis, killing the pathogenic bacteria and viruses, its action mechanism is shown in FIG. 1, and provides a brand new research and development direction for the human need of disinfection. A disinfection system obtained by this new research and development direction is particularly suitable for air purifier system, obviates the need for antibiotics and anti-viral drugs, contains no halogen (e.g., sodium hypochlorite, bromine water, iodine, etc.), contains no peroxides (such as peroxy acid, peroxy alcohol, hydrogen peroxide, peroxy acid salt, such as sodium percarbonate, etc.), contains no ethanol having hidden risk of explosion, and also uses no silver ion with a high use cost, and can be continuously used for a long time along with operation of an air purifier, may not produce secondary pollutants such as organic matter and free radical and ozone etc., may not cause the emergence of drug resistant pathogenic bacteria and viruses, and has low production and use cost, no corrosion and high safety.

Under the direction of this research and development concept, after a disinfection system of anthraquinone salt system had been successfully developed, a disinfection system of bisphenol salt was also invented.

### SUMMARY OF THE INVENTION

The purpose of the present invention is providing an application of bisphenol salt in preparing a disinfectant for sterilization or formaldehyde removal. The present invention provides a new disinfectant system that can be used in an air purifier.

The technical solution adopted by the present invention is:

Application of the bisphenol salt having a structure as shown in Formula (I) and (II) in preparing the disinfectant:

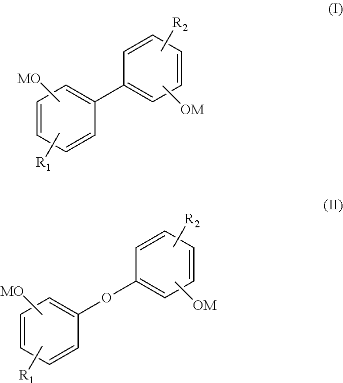

In Formula (I) or (II), R₁, R₂ each individually represents sulfonic group, formyloxy group, halogen atom or hydrogen, and M is potassium, sodium or ammonium.

Preferably, the bisphenol salt is one of the following formulas:

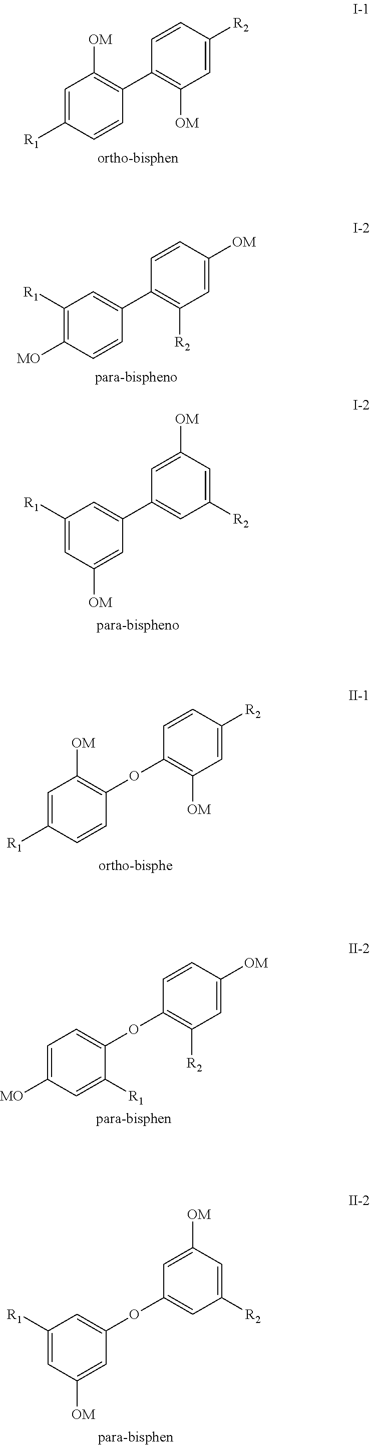

The disinfection efficacy of the bisphenol salts are revealed under oxygen ventilation condition, thus, when used as the disinfectant, it is required to ventilate air continuously. Under normal operating condition of the air purifier (5 minutes or more, room temperature, a large amount of air being ventilated), the pathogenic bacteria and viruses can be effectively killed, in order to achieve the disinfection purpose.

Specifically, the disinfectant is a base solution prepared by evenly dispensing the bisphenol salt into a solvent, and the pH is 8 to 12 (below this range, the disinfection effect will be poor; above this range, the solution will have a certain corrosion), in the presence of oxygen, an effective disinfection effect is obtained; the solvent is water, glycerol or a mixture thereof; the mass concentration of the bisphenol salt in the base solution is 0.5% to 20% (generally the mass concentration is no more than 10%, most preferably less than 5%, if the mass concentration is too high the use cost will increase), such base solution may be directly used as the disinfectant, it may be also add some common additives and then used as the disinfectant, the additives include flavor and pigment etc., without specific limitation, as long as the purpose of the present invention is not influenced. The disinfectant product can be made into a form of liquid, slurry-like paste, and solid etc.

Preferably, the pH of the disinfectant is 9.5 to 11 (the pH is adjusted with NaOH), and the mass concentration of the bisphenol salt in the solvent is 1% to 5%.

The disinfectant is primarily used in being added into the air purifier (for example, the disinfectant solution is put in an air inlet passage of the air purifier), for sterilization or formaldehyde removal.

The beneficial effect of the present invention is primarily embodied in: the disinfectant of the present invention, under the condition for continuously introducing the air, can be used for a long period of time, without need for anti-viral drugs or antibiotics etc., and without need for the presence of the peroxides having hidden safety trouble, without use of the irritant substances containing halogen, and further without use of alcohol with explosive hazard. Such disinfectant system without use of precious metal is easy to prepare, and has low cost of use, does not cause the secondary pollution, and has a good application prospect.

### BRIEF DESCRIPTION OF THE DRAWINGS

FIG. 1 is an action mechanism of killing the microorganisms by oxygen in the air;

FIG. 2 is the results of Kirby-Bauer diffusion detection;

FIG. 3 is a graph for a relationship of the sterilization capability of the bisphenol salt and pH value.

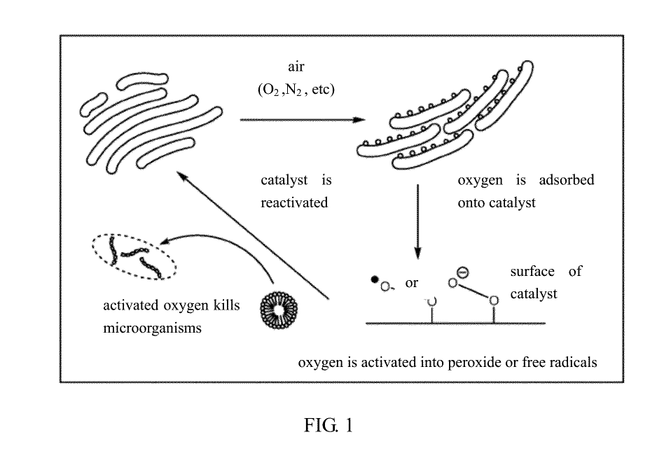

### DETAILED DESCRIPTION OF THE EMBODIMENTS OF THE INVENTION

Hereinafter, the present invention will be further described in conjunction with the specific embodiments, but the protection scope of the present invention is not limited to these.

### EXAMPLE 1

### Catalyst Screening

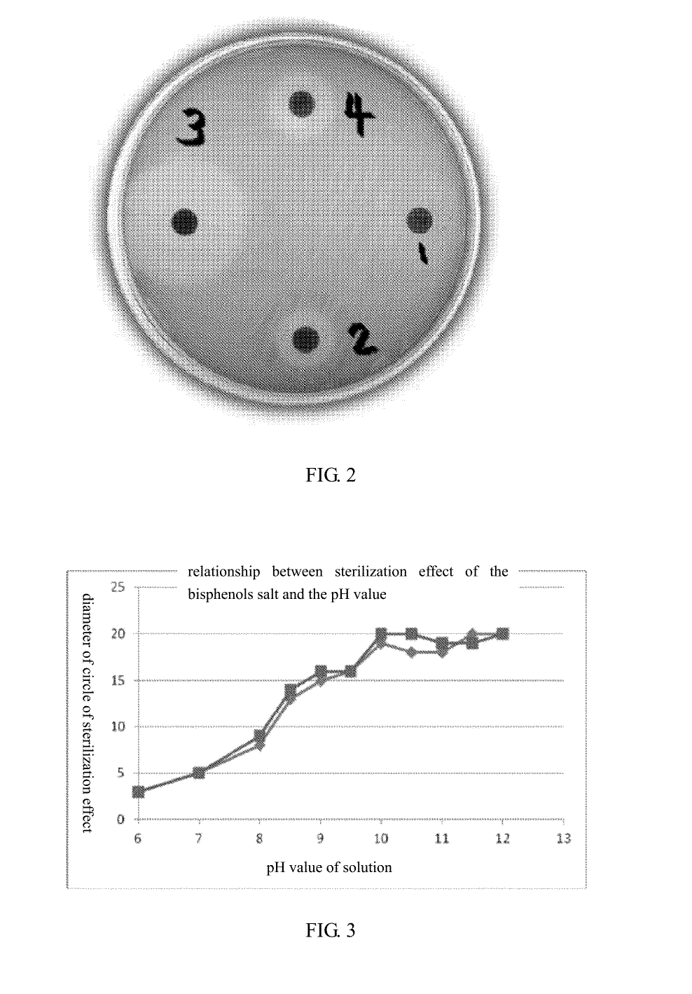

In catalyst screening, Staphylococcus aureus was used as the microorganisms, and a trypsin soybean agar was used as a culture medium. The material powder to be screened was prepared into a homogeneous mixture solution by pure water in a 5% weight/volume ratio (i.e., 5 g powder/100 mL water) (in rare cases of low solubility, firstly dissolved with a small amount of glycerol, then diluted with pure water), then immersed with a neutral precise filtration filter paper of 5 mm diameter for about 10 seconds, then placed into a Kirby-Bauer diffusion detection plate. A Kirby-Bauer diffusion detection plate was placed in a sealed glass cover, and the air was provided into the glass cover at a speed of about 10 liters per minute. The sterilizing capability of the catalyst was measured by the diameter of the zone without bacterial growth around the filter paper. FIG. 2 is a photograph of the Kirby-Bauer diffusion detection plate for a typical catalytic effect (there were four substances on the plate altogether).

The results were shown in FIG. 2. In FIG. 2, No. 1 was water-soluble cellulose, No. 2 was sulfate sodium, No. 3 was 2,2′-ortho-bisphenol sodium, and No. 4 was sodium chloride. As can be seen in the figures, the sterilization effect of the bisphenol salt was very significant (inhibition zone diameter was 15 mm), and the other essentially had no effect; but in case that the air was not introduced, the sterilization effect of the bisphenol salt was very poor (the diameter of the inhibition zone was 2 to 3 mm).

### EXAMPLE 2

A variety of bisphenol salts were respectively prepared into a water solution with a mass concentration being 5%, and the pH was controlled to 10. The sterilization capability was still detected by Kirby-Bauer diffusion detection method in which Staphylococcus aureus was not used as the microorganisms, respectively including two cases of no ventilation of oxygen and ventilation of oxygen. When oxygen was ventilated, the condition for oxygen ventilation was the same, the temperature was room temperature (25□) and 37□. The results are shown in Table 1, the results showed that when the concentration was 5%, at room temperature or somewhat higher temperature, there was no significant difference in the sterilization capability, indicating that such a disinfectant system was stable.

TABLE 1
Sterilization effects at different concentrations and temperatures

| | sterilization effect | | |
|---|---|---|---|
| | without oxygen | sterilization effect | sterilization effect |
| | ventilation | at 25□ | at 37□ |
| bisphenol salts | (diameter, mm) | (diameter, mm) | (diameter, mm) |
| 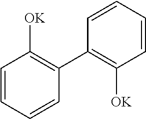 | 3 | 18 | 19 |
| ortho-bisphenol | | | |
| potassium | | | |
| 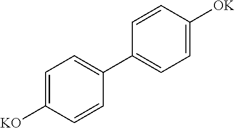 | 2 | 20 | 18 |
| 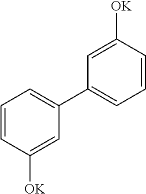 | 2 | 20 | 19 |
| 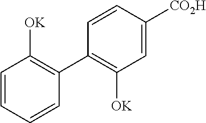 | 2 | 19 | 19 |
| 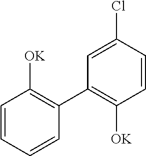 | 3 | 19 | 19 |
| 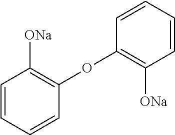 | 5 | 17 | 19 |
| 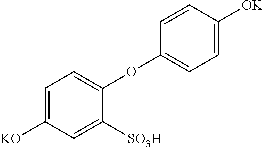 | 6 | 19 | 17 |
| 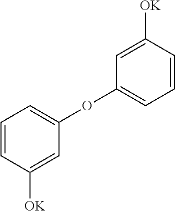 | 6 | 16 | 18 |
| 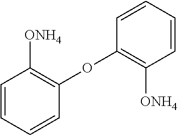 | 5 | 17 | 18 |

The results showed that, for the bisphenol salts, regardless of the location of the substituents, and regardless of Na, NH₄, or K, their capability of catalytically activating oxygen to disinfection were all very good, much higher than the results of no oxygen ventilation.

### EXAMPLE 3

The detail of the test was essentially the same as Example 2, the difference was that the solvent used for dissolving the bisphenol salts was glycerol, the results of the test were essentially identical to Example 2.

### EXAMPLE 4

The ortho-bisphenol potassium was respectively prepared to water solutions with a mass concentration of 0.5%, 1.0%, 2.0%, 5%, 10%, 15%, and 20% (in rare cases of low solubility, firstly dissolved with a small amount of glycerol, then diluted with pure water), and the pH was controlled to 10. The sterilization capability was detected by Kirby-Bauer diffusion detection method in which Staphylococcus aureus was still used as the microorganisms, and the condition for oxygen ventilation was identical, the temperature was room temperature (25□) and 37□. The results were shown in Table 2, indicating that when the concentration was 5% or more, at room temperature or somewhat higher temperature, there was no significant difference in the sterilization capability.

TABLE 2
Sterilization effect at different concentrations and temperatures

| | sterilization effect at | sterilization effect at |
|---|---|---|
| concentration (%) | 25□ (diameter, mm) | 37□ (diameter, mm) |
| 0.5 | 8 | 9 |
| 1.0 | 11 | 10 |
| 2.0 | 14 | 16 |
| 5 | 18 | 19 |
| 10 | 20 | 20 |
| 15 | 19 | 18 |
| 20 | 17 | 19 |

### EXAMPLE 5

### Relationship Between the Sterilization Capability of the Bisphenol Salts and pH Value

Ortho-bisphenol potassium was respectively prepared to form water solutions with a concentration of 5% and pH value of 6, 7, 8, 8.5, 9, 9.5, 10, 10.5, 11, 11.5, 12. The sterilization capability was detected by Kirby-Bauer diffusion detection method in which Staphylococcus aureus was still used as the microorganisms, the condition for oxygen ventilation was identical, the temperature was room temperature (25□) and 37□. The results are shown in Table 3 and FIG. 3, indicating that when the concentration was 5%, at room temperature or somewhat higher temperature, the sterilization capability at pH 8 or more was good, and pH value of 10 or more was the best.

TABLE 3
Data table of relationship between the sterilization effect of the bisphenol
salt and the pH value

| | sterilization effect at | sterilization effect at |
|---|---|---|
| pH | 25□ (diameter, mm) | 37□ (diameter, mm) |
| 6 | 5 | 5 |
| 7 | 5 | 6 |
| 8 | 8 | 9 |
| 8.5 | 13 | 14 |
| 9 | 15 | 16 |
| 9.5 | 16 | 16 |
| 10 | 19 | 20 |
| 10.5 | 18 | 20 |
| 11 | 18 | 19 |
| 11.5 | 20 | 19 |
| 12 | 20 | 20 |

### EXAMPLE 6

### Capability of Removing Formaldehyde by the Disinfectant

In the test, a sealed stainless steel box (0.7 m×0.7 m×0.7 m) was used as a detection space for formaldehyde, and the capability of removing formaldehyde was compared with an activated carbon. For two boxes for test, 500 ml of 5% ortho-bisphenol potassium water solution (pH=10) was put into one of the two boxes, and 500 grams of food-grade activated carbon was put into the other, using formaldehydemeter (PPM Formaldehydemeter 400, the resolution was 0.01 ppm), and change in formaldehyde concentration in the air with an initial formaldehyde concentration being 10 ppm was detected. The results were shown in Table 3.

TABLE 3
Data table of decrease in concentration of formaldehyde in the air

| | effect of ortho-bisphenol | effect of activated |
|---|---|---|
| time (hour) | potassium (ppm) | carbon (ppm) |
| 0 | 10 | 10 |
| 1 | 8.2 | 9.5 |
| 2 | 6.3 | 8.5 |
| 3 | 5.1 | 8.1 |
| 4 | 2.8 | 5.5 |
| 5 | 1.6 | 4.2 |

The results show that, the new disinfection system disclosed in the present invention had a better capability of adsorbing formaldehyde than the activated carbon, indicating that the disinfectant of the present invention will have a good application prospect in air purification field.

The aforementioned are only the preferred embodiments of the present invention, but they are not intended to limit the true scope of the present invention, the true scope of the present invention are broadly defined in the claims of the application, and techniques or methods accomplished by any other person, if they are exactly the same as defined by the claim of the application or an equivalent variation, are all deemed being covered by such claims.

1. A method of preparing a disinfectant comprising the step of utilizing a bisphenol salt of structure of Formula (I) or (II) as follows: 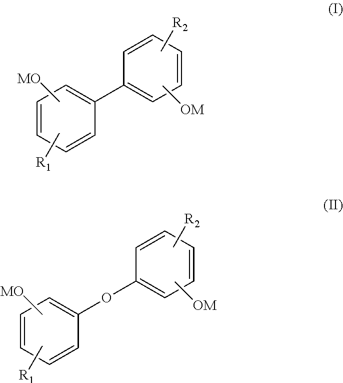
wherein R₁, R₂ each individually represents sulfonic group, formyloxy group, halogen atom or hydrogen, and M is potassium, sodium or ammonium.

2. The method according to claim 1, wherein the bisphenol salt is one of the following formulas:

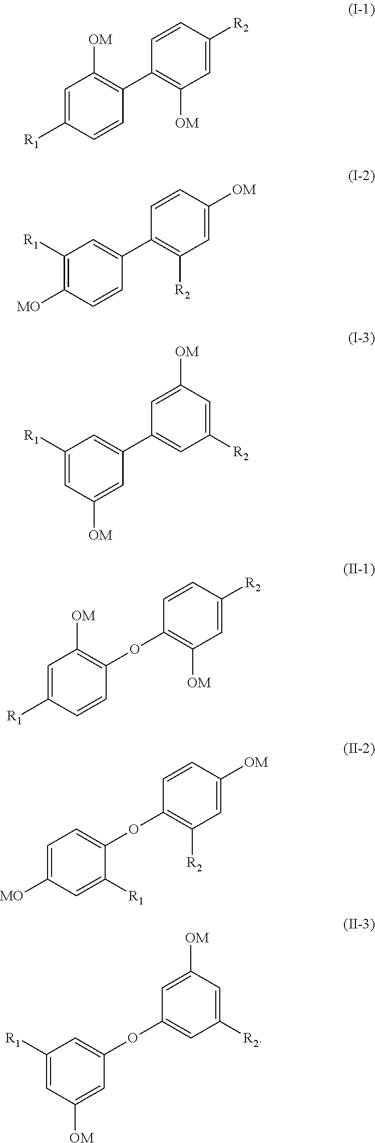

3. The method according to claim 1, wherein the disinfectant is a base solution prepared by evenly dispersing the bisphenol salt into a solvent, and the pH is 8 to 12; the solvent is water, glycerol or a mixture thereof; the mass concentration of the bisphenol salt in the solvent is 0.5% to 20%.

4. The method according to claim 3, wherein the pH value of the base solution is 9.5 to 11, and the mass concentration of the bisphenol salt in the base solution is 1% to 5%.

5. The method according to claim 1, wherein the disinfectant is added into an air purifier, for sterilization or formaldehyde removal.

6. The method according to claim 1, wherein the base solution also comprises additives.

7. The method according to claim 1, wherein the disinfectant is prepared to a liquid, slurry-like paste or solid product.

8. A method of disinfecting an object, comprising the step of applying to the object a disinfectant comprising a bisphenol salt of structure of Formula (I) or (II) as follows: 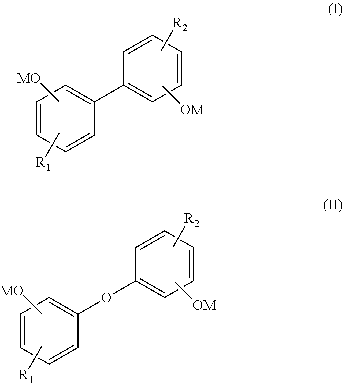
wherein, R₁, R₂ each individually represents sulfonic group, formyloxy group, halogen atom or hydrogen, and M is potassium, sodium or ammonium.

9. The method according to claim 8, wherein the bisphenol salt is one of the following formulas:

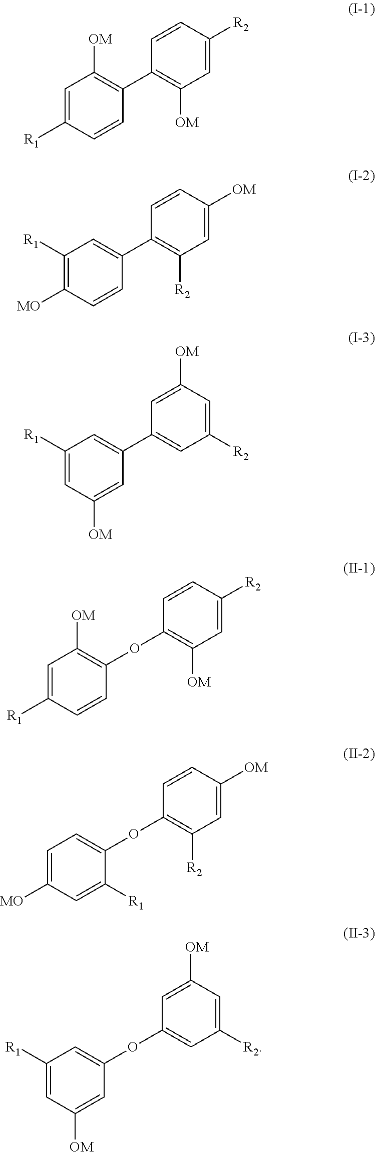

10. The method according to claim 8, wherein the disinfectant is a base solution prepared by evenly dispersing the bisphenol salt into a solvent, and the pH is 8 to 12; the solvent is water, glycerol or a mixture thereof; the mass concentration of the bisphenol salt in the solvent is 0.5% to 20%.

11. The method according to claim 8, wherein the pH value of the base solution is 9.5 to 11, and the mass concentration of bisphenol salt in the base solution is 1% to 5%.

12. The method according to claim 8, wherein the disinfectant is added to an air purifier, for sterilization or formaldehyde removal.

13. The method according to claim 8, wherein the base solution also comprises additives.

14. The method according to claim 8, wherein the disinfectant is prepared to a liquid, a slurry-like paste or a solid product.

15. A disinfectant comprising a bisphenol salt of structure of Formula (I) or (II) as follows: 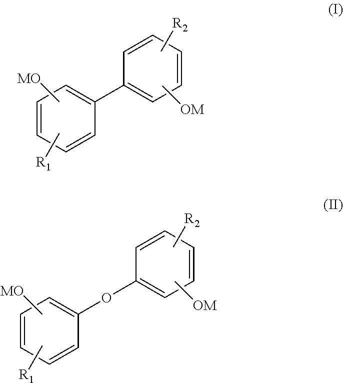
wherein, R₁, R₂ each individually represents sulfonic group, formyloxy group, halogen atom or hydrogen, and M is potassium, sodium or ammonium.

16. The disinfectant according to claim 15, wherein the bisphenol salt is one of the following formulas:

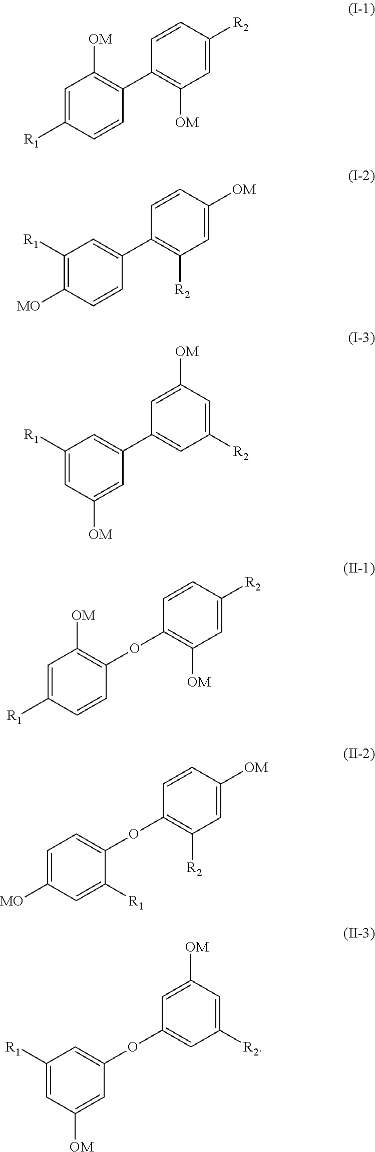

17. The disinfectant according to claim 15, wherein the disinfectant is a base solution prepared by evenly dispersing the bisphenol salt into a solvent, and the pH is 8 to 12; the solvent is water, glycerol or a mixture thereof; the mass concentration of the bisphenol salt in the solvent is 0.5% to 20%.

18. The disinfectant according to claim 15, wherein the pH value of the base solution is 9.5 to 11, and the mass concentration of bisphenol salt in the base solution is 1% to 5%.

19. The disinfectant according to claim 15, wherein the disinfectant is added to an air purifier, for sterilization or formaldehyde removal.

20. The disinfectant according to claim 15, wherein the base solution also comprises additives.
